(19) United States
(12) Patent Application Publication
(10) Pub. No.: US 2009/0035283 A1
(43) Pub. Date: Feb. 5, 2009

(54) Composition and Method for Inhibition of Melanin Synthesis

(75) Inventors: ByungSoon Park, Seoul (KR); WonSerk Kim, Seoul (KR); Jong-Hyuk Sung, Seoul (KR); SeJin Ahn, Seoul (KR)

Correspondence Address: EDWARDS ANGELL PALMER & DODGE LLP, P.O. BOX 55874, BOSTON, MA, 02205, US

(73) Assignee: PROSTEMICS CO., LTD., Seoul (KR)

(21) Appl. No.: 12/180,658

(22) Filed: Jul. 28, 2008

(30) Foreign Application Priority Data
Jul. 30, 2007 (KR) 10-2007-0076221

(51) Int. Cl. A61K 35/12; A61P 17/00

(52) U.S. Cl. 424/93.7

(57) ABSTRACT

The present invention provides a whitening cosmetic composition comprising a stem cell, a culture medium thereof or a protein isolated from the culture medium and a method of whitening a skin which comprises administering to the skin an therapeutically effective amount of a stem cell, a culture medium thereof or a protein isolated from the culture medium.

### CROSS-REFERENCE TO RELATED APPLICATION

This application claims under 35 U.S.C. §119(a) the benefit of Korean Patent Application No. 10-2007-0076221 filed Jul. 30, 2007, the entire contents of which are incorporated herein by reference.

### BACKGROUND

1. Technical Field

The present invention relates to a composition comprising a human adult stem cell or a culture medium thereof and a method of inhibiting melanin synthesis using the stem cell or the culture medium thereof. More particularly, the present invention relates to a whitening cosmetic composition comprising a mesenchymal stem cell, extracted from human adipose, placenta, umbilical cord blood or bone marrow, or a culture medium thereof and a method of inhibiting melanin synthesis using the stem cell or the culture medium thereof.

2. Background Art

Melanin is known to play a key role in determining human skin color. It is produced in a skin cell, called “melanocyte,” and moved to epidermal cells (keratinocyte). It plays various important roles, including forming a cap-like structure around cellular nucleus to protect genes from UV radiation and removing free radicals to protect intracellular proteins.

When there is no melanin-degrading enzyme in the body and when keratinocytes are removed from the epidermis, melanin is removed from the skin. On the other hand, when it is produced more than required, hyperpigmentation, such as melasma, freckles or spots, occurs, leading to unfavorable results in cosmetic terms.

As the number of people who enjoy outdoor activities has increased due to development of leisure industry, demand for preventing melanin pigmentation caused by UV radiation has increased. Whitening agents for preventing excessive production of melanin has been demanded and many efforts have been made.

Majority of such efforts have focused on discovery of materials that can reduce the amount of melanin by inhibiting the activity of tyrosinase, a protein necessary for melanin biosynthesis. Whitening agents developed to date include kojic acid, arbutin, glutathione, vitamin C, vitamin C and the like. They, however, do not have sufficient whitening effect and they have side effects.

Thus, there is a need for a new whitening agent having excellent whitening effect with no or less side effects.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

It is an object of the present invention to provide a whitening agent with improved safety and effectiveness, which contains either a culture medium comprising a stem cell, a culture medium thereof, or a protein isolated from the culture medium.

In one aspect, the present invention provides a whitening cosmetic composition comprising a stem cell culture medium or a protein isolated from the culture medium.

Preferably, the stem cell is a mammalian adult stem cell or mesenchymal stem cell. It may be isolated from adipose tissue, bone marrow tissue or umbilical cord blood.

The above and other features of the invention are discussed infra.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a graph showing the tyrosinase activity inhibitory effect of an adipose-derived stem cell culture, in which AAPE indicates an adipose-derived stem cell culture according to the present invention;

FIG. 2 is a graph showing the mushroom tyrosinase inhibitory activity of the adipose-derived stem cell culture;

FIG. 3 is a graph showing the melanin production inhibitory activity of the adipose-derived stem cell culture;

FIG. 4a shows the results of Western blot of tyrosinase in B16 cells treated with the adipose-derived stem cell culture;

FIG. 4b is a graph showing the inhibition of tyrosinase relative to a marker protein in B16 cells treated with the adipose-derived stem cell culture; and

FIG. 5 is a photograph showing a human skin before and after treatment of the adipose-derived stem cell culture.

### DETAILED DESCRIPTION

The present invention provides a whitening drug or cosmetic product comprising a human adult stem cell or a culture medium thereof and a method of inhibiting melanin synthesis using the stem cell or the culture medium.

The stem cell may comprise a mammalian adult stem cell and a mesenchymal stem cell. The adult stem cell may include a stem cell isolated from adipose tissue, bone marrow tissue, umbilical cord blood or placenta.

As used herein, the term “stem cell” refers to an undifferentiated cell that divides for a long period of time, is capable of self-renewal and can differentiate into diverse cells in predetermined conditions.

Stem cells are classified, according to the tissue from which they originate, into embryonic stem cells and adult stem cells. Adult stem cells have limited potential compared to embryonic stem cells. But they have no ethical problems and side effects. Studies on therapeutic agents have thus been made mainly on adult stem cells.

In the present invention, adult stem cells isolated from adult adipocytes are used. They can be obtained from cells in adipose tissue through a simple purification process as detailed below. According to the present invention, usage of adipose tissue recovered as a waste in a liposuction process is increased, an additional invasive procedure being eliminated.

Stem cells are obtained by collecting human adipose tissue by liposuction under local anesthesia, enzymatically treating the extracellular matrix of the adipose tissue with collagenase, centrifuging the treated tissue and separating monocytes, red blood cells and various cell fragments from the tissue.

Adipose tissue recovered as a waste in a liposuction process, which is generally performed in hospitals, can be collected and isolated in an aseptic state.

The isolated stem cells are cultured in serum-containing Dulbecco's Modified Eagle's Medium (DMEM), and nonadhesive cells are removed from the culture medium.

The stem cells isolated according to the above process are subcultured three times and centrifuged, and the supernatant is filtered, thus obtaining the adipose-derived stem cell culture.

The process of isolating adult stem cells from adipocytes and culturing the isolated cells is not limited to the method disclosed herein and can be carried out according to any conventional methods known in the art.

The adipose-derived stem cells, a culture medium thereof (e.g., a human growth factor-containing culture, produced using mesenchymal adult stem cells), and/or a protein isolated from the culture medium can be effectively used as whitening cosmetic drugs, quasi-drugs, or other cosmetic products for preventing or treating skin pigmentation.

Hereinafter, the present invention will be described in further detail with reference to examples, but the scope of the present invention is not limited to these examples.

### EXAMPLE 1

### Isolation and Culture of Adipose Stem Cells

10 ml of human liposuction material (Leaders Clinic, Seoul, Korea) was washed with the equal volume of phosphate buffer saline, and only adipose tissue was separated from the washed material.

The extracellular matrix of the adipose tissue was enzymatically treated with 0.0075% collagenase in a 5% CO₂ incubator at 37° C. for 45 minutes, and the optimally enzymatically treated adipose tissue was centrifuged at 1200 g for 5 minutes to obtain a stromal vascular fraction containing high-density stem cells. The pellets were washed with phosphate buffer saline and passed through a 70-μm nylon cell filter to remove other tissues, and only cell fragments, including red blood cells, and monocytes, were separated by Histopaque-1077 (SIGMA).

The separated monocytes were cultured in Dulbecco's Modified Eagle's Medium (DMEM), containing 10% fetal bovine serum (FBS) and 1% penicillin streptomycin, in a 5% CO₂ incubator at 37° C. for 24 hours, and then non-adhesive cells were removed therefrom, thus isolating 10⁶ stem cells.

The isolated stem cells were suspended at a concentration of 10⁴ cells/ml, and 10 ml of the cell suspension was transferred to a T25 flask (area: 25 cm², volume: 50 ml) and cultured in the above conditions.

The doubling time was maintained until the cells being cultured in the flask reached a confluence of 80%. At the confluence of 80%, the cells were subcultured.

The subculture was carried out by washing the flask, from which the culture medium has been removed, with PBS, detaching the cells with 0.25% Trypsin-EDTA (GIBCO), centrifuging the cell suspension, measuring the cell count and viability, and then subculturing the cells in a DMEM medium containing, 10% FBS and 1% penicillin streptomycin. The subculture was repeated three times.

### EXAMPLE 2

### Production of Stem Cell Culture Medium

4×10⁵ adipose-derived stem cells, isolated and subcultured in Example 1, were cultured in a serum-free DMEM/F12 medium (Invitrogen-Gibco-BRL, Grand Island, N.Y.) for 72 hours, and then the cell culture medium was centrifuged at 300 g for 5 minutes. The supernatant was filtered with a 0.22-μm injection filter, thus preparing an adipose stem cell culture medium.

### EXAMPLE 3

### Analysis of Proteins in Adipose Stem Cell Culture Medium

3-1: Trypsin Degradation of Proteins

The adipose stem cell culture medium prepared in Example 2 was freeze-dried using a freeze-dryer. The dried powder was dissolved in sterilized distilled water, and proteins were recovered therefrom using a solid-phase extraction cartridge (Waters, USA). The proteins were fractionated into 6 groups using C18 reverse phase chromatography (Chromolith, Merck). Each of the fractions were reduced using reduction buffer (50 mM NH₄HCO₃, 2 mM DTT) at 56° C. for 20 minutes. The reduced proteins were alkylated with alkylation buffer (50 mM NH₄HCO₃, 5 mM iodoacetamide) at 37° C. for 15 minutes, and then degraded with trypsin at 37° C. for 12 hours.

3-2: LC-MS/MS Analysis Using Q-TOF

Each of the peptide fractions degraded with trypsin was analyzed in an Agilent 1100 LC system (Agilent, USA) connected with a Q-STAR Excel mass spectrometer (MDS Sciex, Toronto, Canada). Data were acquired using the information-dependent acquisition mode of Analyst QS software.

The multiple charged ions were screened using MS/MS. Each cycle was composed of 1-s MS and 3-s MS/MS, and the ions were treated using the linear LC at an acetonitrile concentration gradient of 12.5-40% for 90 minutes.

Each of the precursor ions was selected with the tandem MS, and then analyzed using LC-MS/MS.

LC-MS/MS was repeated three times to determine the peptide fraction of each group.

3-3: Database Search

The LC-MS/MS results were searched in the human International Protein Index (IPI) protein sequence database using the MASCOT search engine (Matrix Science, London, United Kingdom). The accuracy of MS was 1200 ppm, the accuracy of MS/MS was 0.3 Da, and O-deoxy-carbamidomethylated cystein was selected as a fixed modification and oxidated methionine as a variable modification. Also, the search was performed at the N-acetylated protein level.

As a result of the database search, a total of 112 proteins were identified, and among them, whitening-related proteins were selected and are shown in Table 1.

TABLE 1
Whitening proteins in adipose stem cell
culture

| Hypopigmenting | | | | |
|---|---|---|---|---|
| mechanism | AC | Gene | MW | Description |
| Tyrosinase, | IPI00007793 | IL6 | 23931 | Interleukin-6 |
| TRP1, TRP2 | | | | precursor |
| inhibitory Mitf | IPI00302679 | LTBP1 | 160801 | Latent transforming |
| down regulation | | | | growth factor beta |
| | | | | binding protein 1 |
| | | | | isoform LTBP-1S |
| | IPI00292150 | LTBP2 | 204059 | Latent transforming |
| | | | | growth factor beta |
| | | | | binding protein 2 |
| | | | | precursor |
| Melanosome | IPI00302679 | LTBP1 | 160801 | Latent transforming |
| transfer | | | | growth factor beta |
| | | | | binding protein 1 |
| | | | | isoform LTBP-1S |
| | IPI00292150 | LTBP2 | 204059 | Latent transforming |
| | | | | growth factor beta |
| | | | | binding protein 2 |
| | | | | precursor |

As described above, it can be seen that the adipose-derived stem cell culture medium contains a number of proteins, which are involved in the inhibition of tyrosinase, TRP1 and TRP2, the downregulation of Mitf (microphthalmia-associated transcription factor and the transfer of melanosome.

### EXAMPLE 4

### Melanin Inhibitory Test in B16 Cell Line

Murine melanoma B16 cell line was cultured in DMEM medium, containing 10% fetal bovine serum, 100 U/ml penicillin and 100 μg/ml streptomycin, in conditions of 37° C. and 5% CO₂.

The cultured B16 cells were inoculated into a 96-well plate at a concentration of 2×10³/well and treated simultaneously with 100 nM melanin-stimulating hormone α-MSH and 200 μl of each of sample solutions, containing each of 0%, 10%, 50% and 100% stem cell cultures. After 72 hours, 10 μl of CCK-8 solution (Dojindo, Gaithersburg, Md.) was added to each well and cultured for 3 hours. Then, the absorbance of each well was measured at 450 nm with a microplate reader (TECAN, Grodig, Austria). The measured values were calibrated with a standard curve.

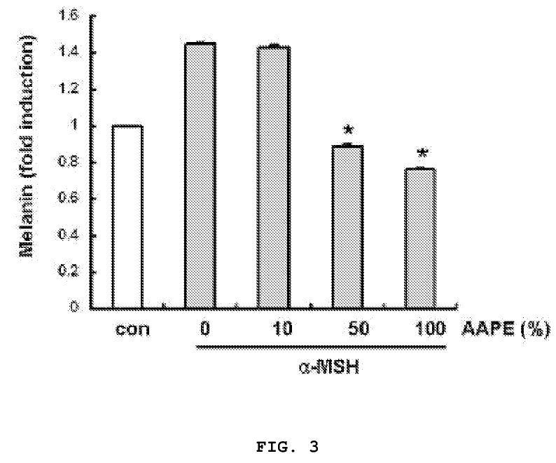

Then, the cultured B16 cells was inoculated into a 96-well plate at a concentration of 1.5×10⁴ cell/well, and then pre-treated with 10 ml of the adipose stem cell culture, obtained in Example 2 and having varying concentrations of 0%, 10%, 50% and 100%, for 1 hour. The pre-treated cells were treated with 100 nM α-MSH for 72 hours, and then the medium was washed with phosphate buffer solution. Then, 100 μl of a melanin extraction solution (1N NaOH+50% DMSO) was added to each well, and the cells were lysed at 80° C. Then, the absorbance of melanin was measured at 492 nm with a microplate reader (see FIG. 3).

As a result, the group treated with 50% or 100% adipose-derived stem cell culture medium together with α-MSH showed excellent melanin production inhibitory effect compared to that of the group not treated with the adipose-derived stem cell culture medium.

### EXAMPLE 5

### Tyrosinase Inhibitory Test

5-1: Tyrosinase Activity Test

220 μl of 0.1 M phosphate buffer saline (pH 6.5), 20 μl of tyrosinase extracted from B16 cells and 20 μl of mushroom tyrosinase were sequentially placed in a test tube. To the solution, 40 μl of 1.5 mM tyrosine solution was added and allowed to react at 37° C. for 10-15 minutes, and the absorbance of the reaction solution was measured at 490 nm with a microplate reader.

Before the solution was treated with the tyrosine solution, it was treated with 200 μl of each of the sample solutions prepared in Example 4, and as a blank sample solution, 0.1 M phosphate buffer solution (pH 6.5) was used.

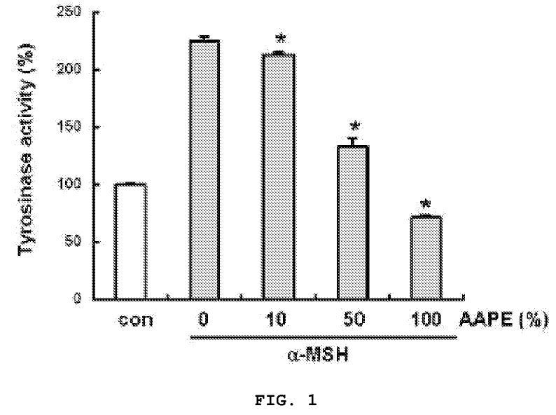

As a result, as shown in FIG. 1, the group treated with the 50% or 100% adipose-derived stem cell culture medium together with α-MSH showed excellent tyrosinase activity inhibitory effect compared to that of the group not treated with the adipose-derived stem cell culture medium.

5-2: Tyrosinase Inhibition Test

850 μl of 0.1 M phosphate buffer saline (pH 7.0), 50 μl of the sample solution of Example 4 and 50 μl of mushroom tyrosinase were sequentially placed in a test tube and allowed to react at 37° C. for 6 minutes. To the solution, 50 μl of 0.06 mM L-DOPA (L-3,4-dihydroxyphenylalanine) solution was added and, then allowed to react at 37° C. for 1 minute. As a blank sample solution, 0.1 M phosphate buffer saline (pH 7.0) was used. After completion of the test, the absorbance of the reaction solution was measured at 475 nm with a microplate reader.

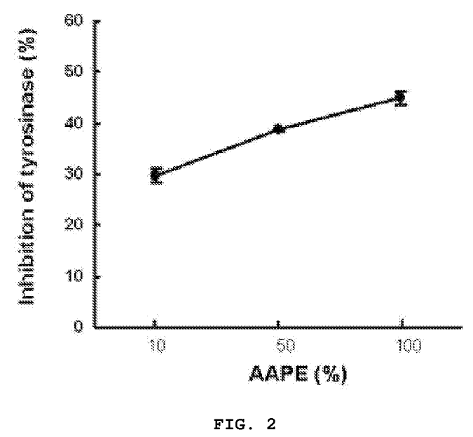

As a result, as shown in FIG. 2, the activity of tyrosinase in the group treated with the stem cell culture medium according to the present invention was inhibited in proportion with the concentration of the stem cell culture medium.

### EXAMPLE 6

### Western Blot Analysis of Tyrosinase

The cultured B16 cells were inoculated into a 96-well plate at a concentration of 1.5×10⁵ cells/well, and then 10 ml of each of 0%, 10%, 50% and 100% adipose stem cell cultures for 2 hours. Then, the cells were treated with 10 ml of the sample solution of Example 4.

After 48 hours, the cells were lysed with RIPA buffer (50 mM Tris-HCl, 0.15 M NaCl, 1 mM EDTA, 1% Triton X-100, 1% SDS, 50 mM NaF, 1 mM Na₃VO₄, 5 mM dithiothreitol, 1 μg/ml leupeptin and 20 μg/ml PMSF, pH 7.4), thus obtaining a protein. 25 mg of the protein was separated using 8% SDS-polyacryamide gel electrophoresis. The gel having the separated protein thereon was transferred to a PVDF membrane. The PVDF membrane was incubated with anti-tyrosinase (1:500 dilution) antibody and α-tubulin antibody (1:10,000 dilution), and then incubated with horseradish peroxidase-conjugated anti-goat IgG antibody (1:10,000 dilution). The protein bands were detected using immunobilon western reagent and exposed to X-ray film.

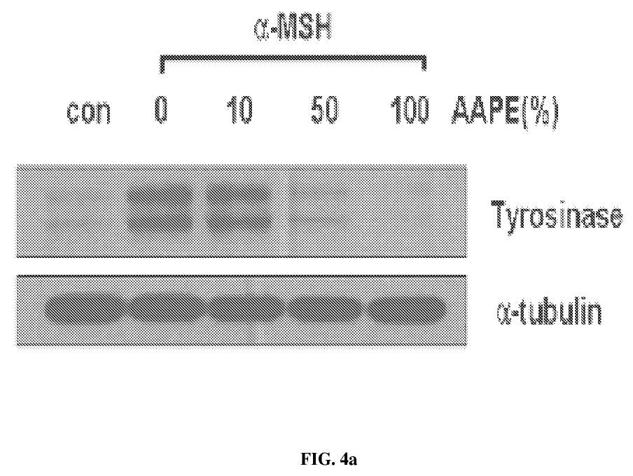

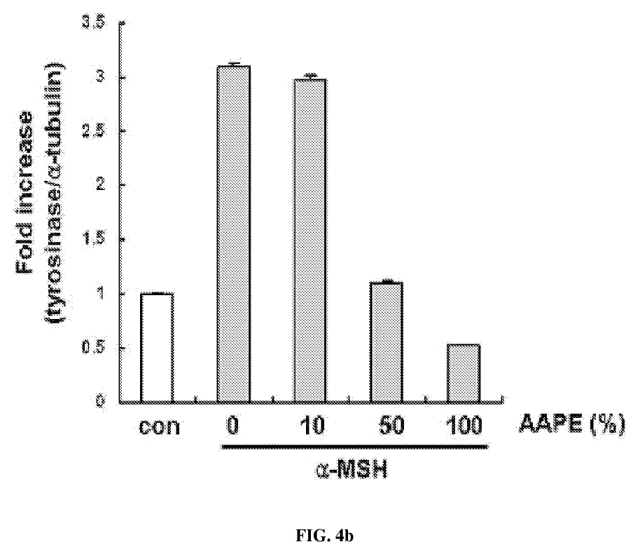

As a result, as shown in FIG. 4a, no tyrosinase was detected in the group treated with the 50% or 100% adipose stem cell culture according to the present invention, and as can be seen in FIG. 4b, the expression of tyrosinase in the treated group was significantly inhibited.

### EXAMPLE 7

### Whitening Effect Resulting from Application of Adipose-Derived Stem Cell Culture

This test was a pilot study which was conducted under the approval of the Institutional Review Board of Kangbuk Samsung Medical Center (Seoul, Republic of Korea). The object of this test is to evaluate the whitening effect of the adipose-derived stem cell culture in patients in which skin aging is in progress.

This test was conducted on healthy volunteers consisting of adult men and women, who were 20-75 years of age and desire whitening improvement due to pigmented lesions.

Specifically, 6 ml of the adipose stem cell culture separated in Example 2 was prepared, and it was uniformly applied on the portions that the patients desired improvement, and was then rubbed with microneedles. According to this method, the adipose stem cell culture was applied once at a 2-week interval, a digital photograph of the applied portion was observed, and the whitening effect of the stem cell culture medium was measured. The application of the adipose stem cell culture medium was repeated three times or more.

In order to evaluate the effectiveness of the adipose stem cell culture, a digital photograph of the applied portion was taken, and the effect of the stem cell culture was evaluated based on the scores shown in Table 2 below.

TABLE 2
Scores for evaluating effect

| Scores | Criteria |
|---|---|
| 4: very good | Improved by more than 76% |
| 3: good | Improved by 51-75% |
| 2: moderate | Improved by 26-50% |
| 1: bad | Improved by less than 25% |
| 0: worse | Not changed or became more |
| | severe |

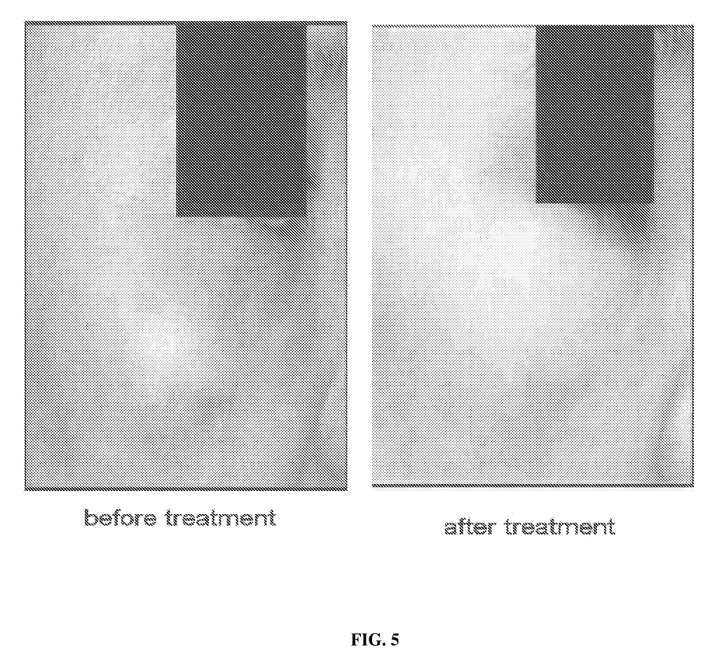

As a result, as can be seen in FIG. 5, the adipose-derived stem cell culture could reduce skin darkening. Also, as shown in Table 3 below, when the adipose-derived stem cell culture medium was administered, 87.9% of the subjects showed an improvement of more than 26%.

TABLE 3
Results of administration of adipose-derived stem
cell line

| | Degree of improvement | | | | | |
|---|---|---|---|---|---|---|
| | | | | | Very | |
| | Worse | Bad | Moderate | Good | good | Total |
| Number of | 3 | 8 | 25 | 41 | 14 | 91 |
| subjects | | | | | | |
| Percentage | 3.30% | 8.79% | 27.47% | 45.05% | 15.38% | 100.00% |

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A whitening cosmetic composition comprising a stem cell, a culture medium thereof or a protein isolated from the culture medium.

2. The composition of claim 1, wherein the stem cell is a mammalian adult stem cell or mesenchymal stem cell.

3. The composition of claim 2, wherein the stem cell is isolated from the group consisting of adipose tissue, bone marrow tissue and umbilical cord blood.

4. A method of whitening a skin which comprises administering to the skin a therapeutically effective amount of a stem cell, a culture medium thereof or a protein isolated from the culture medium.

5. The method of claim 4, wherein the stem cell is a mammalian adult stem cell or mesenchymal stem cell.

6. The method of claim 5, wherein the adult stem cell is isolated from the group consisting of adipose tissue, bone marrow tissue and umbilical cord blood.
